(19) United States
(12) Patent Application Publication
(10) Pub. No.: US 2011/0046231 A1
(43) Pub. Date: Feb. 24, 2011

(54) SOLID FORMS OF (±)-O-DESMETHYLVENLAFAXINE SALTS

(75) Inventors: Sonny Sebastian, Cherthala (IN); Raman Buddhavarapu Pattabhi, Hyderabad (IN); Krishna Challa, Hyderabad (IN); Nitin Sharadchandra Pradhan, Maharashtra (IN); Jon Valgeirsson, Hafnarfjordur (IS)

Correspondence Address: CANTOR COLBURN LLP, 20 Church Street, 22nd Floor, Hartford, CT, 06103, US

(73) Assignee: ACTAVIS GROUP PTC EHF, 220 Hafnarfjordur (IS)

(21) Appl. No.: 12/739,163

(22) PCT Filed: Oct. 21, 2008
(86) PCT No.: PCT/IB08/03308

§ 371 Date: Sep. 13, 2010

(30) Foreign Application Priority Data
Oct. 22, 2007 (IN) 2374/CHE/2007

(51) Int. Cl. A61K 31/137; C07C 217/74

(52) U.S. Cl. 514/653; 564/355

(57) ABSTRACT

The present invention relates to solid forms of (±)-O-desmethylvenlafaxine salts, processes for preparation, pharmaceutical compositions, and method of treating thereof. More particularly, the present invention provides solid forms of acid addition salts of (±)-O-desmethylvenlafaxine wherein the acid counter ion is provided by an acid selected from the group consisting of oxalic acid, benzoic acid and lactic acid.

### CROSS REFERENCE TO RELATED APPLICATION

This application claims the benefit of priority to Indian provisional application No. 2374/CHE/2007, filed on Oct. 22, 2007, which is incorporated herein by reference.

### FIELD OF THE DISCLOSURE

The present invention relates to solid forms of (±)-O-desmethylvenlafaxine salts, processes for preparation, pharmaceutical compositions, and method of treating thereof. More particularly, the present invention provides solid forms of acid addition salts of (±)-O-desmethylvenlafaxine wherein the acid counter ion is provided by an acid selected from the group consisting of oxalic acid, benzoic acid and lactic acid.

### BACKGROUND OF THE INVENTION

O-Desmethylvenlafaxine, chemically named (±)-1-[2-(dimethylamino)-1-(4-hydroxyphenyl)ethyl]cyclohexanol, is a major metabolite of venlafaxine and has been shown to inhibit norepinephrine and serotonin uptake. O-Desmethylvenlafaxine, which can also be referred to as desvenlafaxine or desmethylvenlafaxine, is represented by the following structural formula:

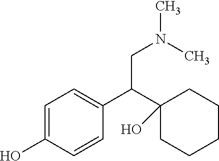

and its first synthesis was disclosed in U.S. Pat. No. 4,535,186 (hereinafter referred to as the '186 patent). Salts of O-desmethylvenlafaxine, including the fumarate, succinate, formate, maleate, tartrate and citrate salts, have been described in the literature. O-Desmethylvenlafaxine has been exemplified as a fumarate salt in the '186 patent, and a succinate and formate salts were disclosed in U.S. Pat. Nos. 6,673,838 and 7,001,920, respectively. O-Desmethylvenlafaxine is also exemplified as a free base in PCT publication No. WO 00/32555.

While the '186 patent generally mentions that the basic compounds disclosed in that patent can form a salt with pharmaceutically acceptable organic or inorganic acids like hydrochloric acid, hydrobromic acid, maleic acid, fumaric acid, succinic acid, sulfuric acid, phosphoric acid, tartaric acid, acetic acid, citric acid, oxalic acid and similar acids, only the fumarate salt of O-desmethylvenlafaxine has been prepared and isolated.

PCT publication No. WO 2008/103461 discloses solid forms comprising stereomerically pure (−)-O-desmethylvenlafaxine, including salts thereof, particularly hydrochloride salt, compositions comprising the solid forms, methods of making the solid forms and methods of use thereof.

In the formulation of drug compositions, it is important for the active pharmaceutical ingredient to be in a form in which it can be conveniently handled and processed. This is of critical, not only from the point of view of obtaining a commercially viable manufacturing process, but also from the point of view of subsequent manufacture of pharmaceutical formulations (e.g. oral dosage forms such as tablets) comprising the active pharmaceutical ingredient.

Further, in the manufacture of oral pharmaceutical compositions, it is important that a reliable, reproducible and constant plasma concentration profile of the active pharmaceutical ingredient is provided following administration to a patient.

Chemical stability, solid state stability, and “shelf life” of the active pharmaceutical ingredient are important properties for a pharmaceutical active compound. The active pharmaceutical ingredient, and compositions containing it, should be capable of being effectively stored over appreciable periods of time, without exhibiting a significant change in the physico-chemical characteristics of the active pharmaceutical ingredient, e.g. its chemical composition, density, hygroscopicity and solubility. Thus, in the manufacture of commercially viable and pharmaceutically acceptable drug compositions, it is important, wherever possible, to provide the active pharmaceutical ingredient in a stable form.

New solid forms of a pharmaceutical agent can further the development of formulations for the treatment of illnesses. For instance, solid forms of salts of a compound are known in the pharmaceutical art to affect, for example, the solubility, dissolution rate, bioavailability, chemical and physical stability, flowability, tractability, and compressibility of the compound as well as the safety and efficacy of drug products based on the compound.

Polymorphism is the occurrence of different crystalline forms of a single compound and it is a property of some compounds and complexes. Thus, polymorphs are distinct solids sharing the same molecular formula, yet each polymorph may have distinct physical properties. Therefore, a single compound may give rise to a variety of polymorphic forms where each form has different and distinct physical properties, such as different solubility profiles, different melting point temperatures and/or different x-ray diffraction peaks. Solvent medium and mode of crystallization play very important role in obtaining a new salt or a crystalline form over the other.

The discovery of novel solid forms, including amorphous forms and crystal forms, of pharmaceutically useful compound provides a new opportunity to improve the performance characteristics of a pharmaceutical product. It also adds value to the material that a formulation scientist can use the same for designing, for example, a pharmaceutical dosage form of a drug with a targeted release profile or other desired characteristic. Novel solid forms, including amorphous forms and crystal forms, of (±)-O-desmethylvenlafaxine salts have now been discovered.

### SUMMARY OF THE INVENTION

According to one aspect of the present invention, there is provided novel solid state forms, including amorphous forms and crystalline forms, of (±)-O-desmethylvenlafaxine salts, wherein the salt is selected from the group consisting of oxalate, benzoate and lactate.

In another aspect, (±)-O-desmethylvenlafaxine salts in a solid state is provided. In another aspect, (±)-O-desmethylvenlafaxine salts in a crystalline form is provided. In yet another aspect, (±)-O-desmethylvenlafaxine salts in an amorphous form is provided. In another aspect, the solid state forms of (±)-O-desmethylvenlafaxine salts may exist in an anhydrous and/or solvent-free form or as a hydrate and/or a solvate form.

It has also been found that the novel solid forms of (±)-O-desmethylvenlafaxine salts are useful intermediates in the preparation of (±)-O-desmethylvenlafaxine free base or a pharmaceutically acceptable salt thereof in high purity. The solid forms of (±)-O-desmethylvenlafaxine salts have good flow properties and are far more stable at room temperature, enhanced temperature and at relative high humidities and in aqueous media, and so, the novel solid salt forms are suitable for formulating (±)-O-desmethylvenlafaxine.

In another aspect, the present invention encompasses a process for preparing the novel solid forms of (±)-O-desmethylvenlafaxine salts comprising contacting (±)-O-desmethylvenlafaxine free base with a suitable acid in a suitable solvent under suitable conditions, and isolating the appropriate salt forms of (±)-O-desmethylvenlafaxine as solid, wherein the suitable acid is selected from the group consisting of oxalic acid, benzoic acid and lactic acid.

The suitable solvent is selected from the group comprising water, alcohols, ketones, chlorinated hydrocarbons, hydrocarbons, nitriles, esters, ethers, polar aprotic solvents, and mixtures thereof. Preferable solvents are alcohols, ethers, hydrocarbons, chlorinated hydrocarbons, nitriles, and mixtures thereof, and most preferably methanol, ethanol, isopropanol, methylene chloride, and mixtures thereof.

In another aspect, the present invention provides a method for the use of novel solid forms of (±)-O-desmethylvenlafaxine salts for the treatment of patients suffering from depression (e.g., major depressive disorder, bipolar disorder, and dysthymia), anxiety, panic disorder, generalized anxiety disorder, post traumatic stress disorder, attention deficit disorder, obsessive compulsive disorder, schizophrenia, obesity, anorexia nervosa, cognitive enhancement, cognitive impairment, and cessation of smoking; comprising administering the novel solid forms of (±)-O-desmethylvenlafaxine salts, or a pharmaceutical composition that comprises novel solid forms of (±)-O-desmethylvenlafaxine salts, along with pharmaceutically acceptable excipients.

In another aspect, the present invention provides pharmaceutical compositions comprising a therapeutically effective amount of the solid forms of (±)-O-desmethylvenlafaxine salts of the present invention, and one or more pharmaceutically acceptable excipients.

In another aspect, the present invention provides pharmaceutical compositions comprising the solid forms of (±)-O-desmethylvenlafaxine salts prepared according to processes of the present invention in any of its embodiments and one or more pharmaceutically acceptable excipients.

In yet another aspect, the present invention further encompasses a process for preparing a pharmaceutical formulation comprising combining any one of the solid forms of (±)-O-desmethylvenlafaxine salts prepared according to processes of the present invention in any of its embodiments, with one or more pharmaceutically acceptable excipients.

In another aspect, the substantially pure solid forms of (±)-O-desmethylvenlafaxine salts disclosed herein for use in the pharmaceutical compositions of the present invention, wherein 90 volume-percent of the particles (D₉₀) have a size of less than or equal to about 500 microns, specifically less than or equal to about 300 microns, more specifically less than or equal to about 200 microns, still more specifically less than or equal to about 100 microns, and most specifically less than or equal to about 15 microns.

Unless otherwise indicated, the following definitions are set forth to illustrate and define the meaning and scope of the various terms used to describe the invention herein.

The term “solid form of (±)-O-desmethylvenlafaxine salts disclosed herein” includes crystalline forms, amorphous form, hydrated and solvated forms of (±)-O-desmethylvenlafaxine salts.

The term “crystalline polymorph” refers to a crystal modification that can be characterized by analytical methods such as X-ray powder diffraction, IR-spectroscopy, differential scanning calorimetry (DSC) or by its melting point.

The term “amorphous” means a solid without long-range crystalline order. Amorphous form of (±)-O-desmethylvenlafaxine salts in accordance with the present invention preferably contains less than about 10% crystalline forms of (±)-O-desmethylvenlafaxine salt, more preferably less than 5% crystalline form of (±)-O-desmethylvenlafaxine salt, and still more preferably is essentially free of crystalline forms of (±)-O-desmethylvenlafaxine salt. “Essentially free of crystalline forms of (±)-O-desmethylvenlafaxine salt” means that no crystalline polymorph forms of (±)-O-desmethylvenlafaxine salt can be detected within the limits of a powder X-ray diffractometer.

The term “pharmaceutically acceptable” means that which is useful in preparing a pharmaceutical composition that is generally non-toxic and is not biologically undesirable and includes that which is acceptable for veterinary use and/or human pharmaceutical use.

The term “pharmaceutical composition” is intended to encompass a drug product including the active ingredient(s), pharmaceutically acceptable excipients that make up the carrier, as well as any product which results, directly or indirectly, from combination, complexation or aggregation of any two or more of the ingredients. Accordingly, the pharmaceutical compositions of the present invention encompass any composition made by admixing the active ingredient, active ingredient dispersion or composite, additional active ingredient(s), and pharmaceutically acceptable excipients.

The expression “pharmaceutically acceptable salt” is meant those salts which are, within the scope of sound medical judgment, suitable for use in contact with the tissues of humans and lower animals without undue toxicity, irritation, allergic response and the like, commensurate with a reasonable benefit/risk ratio, and effective for their intended use.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host causing a therapeutically effective blood concentration of the active ingredient at the particular location. This can be accomplished, e.g., by topical, local or by systemic administration of the active ingredient to the host.

The term “buffering agent” as used herein is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

The term “sweetening agent” as used herein is intended to mean a compound used to impart sweetness to a formulation. Such compounds include, by way of example and without limitation, aspartame, dextrose, glycerin, mannitol, saccharin sodium, sorbitol, sucrose, fructose and other such materials known to those of ordinary skill in the art.

The term “binders” as used herein is intended to mean substances used to cause adhesion of powder particles in granulations. Such compounds include, by way of example and without limitation, acacia alginic acid, tragacanth, carboxymethylcellulose sodium, polyvinylpyrrolidone, compressible sugar (e.g., NuTab), ethylcellulose, gelatin, liquid glucose, methylcellulose, povidone and pregelatinized starch, combinations thereof and other material known to those of ordinary skill in the art. If required, other binders may also be included in the present invention.

Exemplary binders include starch, polyethylene glycol, guar gum, polysaccharide, bentonites, sugars, invert sugars, poloxamers (PLURONIC™ F68, PLURONIC™ F127), collagen, albumin, celluloses in nonaqueous solvents, combinations thereof and the like. Other binders include, for example, polypropylene glycol, polyoxyethylene-polypropylene copolymer, polyethylene ester, polyethylene sorbitan ester, polyethylene oxide, microcrystalline cellulose, polyvinylpyrrolidone, combinations thereof and other such materials known to those of ordinary skill in the art.

The term “diluent” or “filler” as used herein is intended to mean inert substances used as fillers to create the desired bulk, flow properties, and compression characteristics in the preparation of solid dosage formulations. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

The term “glidant” as used herein is intended to mean agents used in solid dosage formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

The term “lubricant” as used herein is intended to mean substances used in solid dosage formulations to reduce friction during compression of the solid dosage. Such compounds include, by way of example and without limitation, calcium stearate, magnesium stearate, mineral oil, stearic acid, zinc stearate, combinations thereof and other such materials known to those of ordinary skill in the art.

The term “disintegrant” as used herein is intended to mean a compound used in solid dosage formulations to promote the disruption of the solid mass into smaller particles which are more readily dispersed or dissolved. Exemplary disintegrants include, by way of example and without limitation, starches such as corn starch, potato starch, pregelatinized, sweeteners, clays, such as bentonite, macrocrystalline cellulose (e.g. Avicel™), carsium (e.g. Amberlite™), alginates, sodium starch glycolate, gums such as agar, guar, locust bean, karaya, pectin, tragacanth, combinations thereof and other such materials known to those of ordinary skill in the art.

The term “wetting agent” as used herein is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxyl propylcellulose, hydroxypropylmethylcellulose phthalate, noncrystalline cellulose, magnesium aluminum silicate, triethanolamine, polyvinyl alcohol, and polyvinylpyrrolidone (PVP). Tyloxapol (a nonionic liquid polymer of the alkyl aryl polyether alcohol type, also known as superinone or triton) is another useful wetting agent, combinations thereof and other such materials known to those of ordinary skill in the art.

As used herein, D_{X} means that X percent of the particles have a diameter less than a specified diameter D. Thus, a D₉₀ of less than 300 microns means that 90 volume-percent of the micronized particles in a composition have a diameter less than 300 microns.

The term “micronization” used herein means a process or method by which the size of a population of particles is reduced.

As used herein, the term “micron” or “μm” both are same refers to “micrometer” which is 1×10⁻⁶ meter.

As used herein, “crystalline particles” means any combination of single crystals, aggregates and agglomerates.

As used herein, “Particle Size Distribution (P.S.D)” means the cumulative volume size distribution of equivalent spherical diameters as determined by laser diffraction in Malvern Master Sizer 2000 equipment or its equivalent. “Mean particle size distribution, i.e., D₅₀” correspondingly, means the median of said particle size distribution.

The term “water content” refers to the content of water based upon the Loss on Drying method as described in Pharmacopeial Forum, Vol. 24, No. 1, page 5438 (January-February 1998), the Karl Fisher assay for determining water content or thermogravimetric analysis (TGA). The calculation of water content is based upon the percent of weight that is lost by drying.

The term “Anti-solvent” refers to a solvent which when added to an existing solution of a substance reduces the solubility of the substance.

The term “alcohol solvents” include, but are not limited to, C₁ to C₈ straight or branched chain alcohol solvents such as methanol, ethanol, n-propanol, isopropanol, n-butanol, isobutanol, tert-butanol, amyl alcohol, hexanol, and mixtures thereof.

The term “ketone solvents” include, but are not limited to, acetone, methyl ethyl ketone, methyl isobutyl ketone, methyl tert-butyl ketone and the like, and mixtures thereof.

The term “nitrile solvents” include, but are not limited to, acetonitrile and the like, and mixtures thereof.

The term “ester solvents” include, but are not limited to, ethyl acetate, isopropyl acetate, and the like and mixtures thereof.

The term “chlorinated hydrocarbon solvents” include, but are not limited to, methylene chloride, ethyl dichloride, chloroform, carbon tetrachloride, and mixtures thereof.

The term “cyclic ether solvents” include, but are not limited to, tetrahydrofuran, dioxane, and the like, and mixtures thereof.

The term “aliphatic ether solvents” include, but are not limited to, diethyl ether, diisopropyl ether, monoglyme, diglyme and the like, and mixtures thereof.

The term “hydrocarbon solvents” means both aliphatic and aromatic hydrocarbon solvents include, but are not limited to, n-pentane, n-hexane, n-heptane and isomers thereof, cyclohexane, toluene and xylene and the like, and mixtures thereof.

The term “polar aprotic solvents” include, but are not limited to, N,N-dimethylformamide, N,N-dimethylacetamide, dimethylsulfoxide, and mixtures thereof.

By “substantially pure” is meant having purity greater than about 98%, specifically greater than about 99%, more specifically greater than about 99.5%, and still more specifically greater than about 99.9% measured by HPLC.

### BRIEF DESCRIPTION OF THE DRAWINGS

FIG. 1 is a characteristic powder X-ray diffraction (XRD) pattern of crystalline (±)-O-desmethylvenlafaxine oxalate.

FIG. 2 is a characteristic differential scanning calorimetric (DSC) thermogram of crystalline (±)-O-desmethylvenlafaxine oxalate.

FIG. 3 is a characteristic powder X-ray diffraction (XRD) pattern of crystalline (±)-O-desmethylvenlafaxine benzoate.

FIG. 4 is a characteristic differential scanning calorimetric (DSC) thermogram of crystalline (±)-O-desmethylvenlafaxine benzoate.

FIG. 5 is a characteristic powder X-ray diffraction (XRD) pattern of crystalline (±)-O-desmethylvenlafaxine lactate.

FIG. 6 is a characteristic differential scanning calorimetric (DSC) thermogram of crystalline (±)-O-desmethylvenlafaxine lactate.

The X-Ray powder diffraction was measured by an X-ray powder Diffractometer equipped with CuKα-radiations (40 kV, 40 mA) in wide-angle X-ray Diffractometer of BRUKER axs, D8 ADVANCE. The sample was analyzed using the following instrument parameters: measuring range=3-50° 2-theta; step width=0.01579°; and measuring time per step=0.11 sec.

The DSC thermogram was measured on Perkin—Elmer jade-DSC differential scanning colorimeter in a temperature range of 10-350° C. with a heating rate of 10° C./minute.

### DETAILED DESCRIPTION OF THE INVENTION

According to one aspect of the present invention, there is provided novel solid state forms, including amorphous forms and crystalline forms, of (±)-O-desmethylvenlafaxine salts, wherein the salt is selected from the group consisting of oxalate, benzoate and lactate.

In another aspect, (±)-O-desmethylvenlafaxine salts in a solid state is provided. In another aspect, (±)-O-desmethylvenlafaxine salts in a crystalline form is provided. In yet another aspect, (±)-O-desmethylvenlafaxine salts in an amorphous form is provided. In another aspect, the solid state forms of (±)-O-desmethylvenlafaxine salts may exist in an anhydrous and/or solvent-free form or as a hydrate and/or a solvate forms.

In another embodiment, the novel solid forms of (±)-O-desmethylvenlafaxine salts are useful intermediates in the preparation of (±)-O-desmethylvenlafaxine free base or a pharmaceutically acceptable salt thereof in high purity. The solid forms of (±)-O-desmethylvenlafaxine salts have good flow properties and are far more stable at room temperature, enhanced temperature and at relative high humidities and in aqueous media, and so, the novel solid salt forms are suitable for formulating (±)-O-desmethylvenlafaxine.

According to another aspect of the present invention, a process for the preparation of solid state forms of (±)-O-desmethylvenlafaxine salts is provided, wherein the salt is selected from the group consisting of oxalate, benzoate and lactate; which comprises:
a) contacting (±)-O-desmethylvenlafaxine free base with a suitable acid in a suitable solvent to produce a reaction mass containing (±)-O-desmethylvenlafaxine acid addition salt;
b) optionally, heating the reaction mass obtained in step-(a);
c) optionally, substantially removing the solvent from the reaction mass obtained in step-(a) or step-(b); and/or
d) isolating solid form of (±)-O-desmethylvenlafaxine salt by forcible or spontaneous crystallization;

wherein the suitable acid used in step-(a) is selected from the group consisting of oxalic acid, benzoic acid and lactic acid.

The suitable solvent is selected from the group comprising water, alcohols, ketones, chlorinated hydrocarbons, hydrocarbons, nitriles, esters, ethers, polar aprotic solvents, and mixtures thereof. Preferable solvents are alcohols, ethers, hydrocarbons, chlorinated hydrocarbons, nitriles, and mixtures thereof, and most preferably methanol, ethanol, isopropanol, methylene chloride, and mixtures thereof.

Exemplary alcohol solvents include, but are not limited to, C₁ to C₈ straight or branched chain alcohol solvents such as methanol, ethanol, n-propanol, isopropanol, n-butanol, isobutanol, tert-butanol, amyl alcohol, hexanol, and mixtures thereof. Specific alcohol solvents are methanol, ethanol, isopropanol, tert-butanol and mixtures thereof, and most specific alcohol solvent is isopropanol. Exemplary ketone solvents include, but are not limited to, acetone, methyl ethyl ketone, methyl isobutyl ketone, methyl tert-butyl ketone and the like, and mixtures thereof. Exemplary nitrile solvents include, but are not limited to, acetonitrile, propionitrile and the like, and mixtures thereof. Exemplary ester solvents include, but are not limited to, ethyl acetate, isopropyl acetate, and the like and mixtures thereof. Exemplary chlorinated hydrocarbon solvents include, but are not limited to, methylene chloride, ethyl dichloride, chloroform and carbon tetrachloride or mixtures thereof. Specific chlorinated hydrocarbon solvent is methylene chloride. Exemplary ether solvents include, but are not limited to, diisopropyl ether, diethyl ether, tetrahydrofuran, dioxane, monoglyme, diglyme and the like, and mixtures thereof. Exemplary hydrocarbon solvents include, but are not limited to, n-pentane, n-hexane, n-heptane and isomers thereof, cyclohexane, toluene and xylene and the like, and mixtures thereof. Exemplary polar aprotic solvents include, but are not limited to, N,N-dimethylformamide, N,N-dimethylacetamide, dimethylsulfoxide, and mixtures thereof.

The reaction in step-(a) is carried out at a temperature of about 0° C. to about 100° C., preferably at about 0° C. to about 80° C., more preferably at about 20° C. to about 60° C.

The reaction mass in step-(b) is preferably heated at a temperature of about 40° C. to the reflux temperature of the solvent used for at least 20 minutes, and more preferably at the reflux temperature of the solvent used from about 30 minutes to about 5 hours.

Removal of solvent in step-(c) is accomplished by, for example, substantially complete evaporation of the solvent, concentrating the solution and filtering the solid under inert atmosphere. Alternatively, the solvent may also be removed by evaporation. Evaporation can be achieved at sub-zero temperatures by the lyophilisation or freeze-drying technique. The solution may also be completely evaporated in, for example, a pilot plant Rota vapor, a Vacuum Paddle Dryer or in a conventional reactor under vacuum above about 720 mm Hg by flash evaporation techniques by using an agitated thin film dryer (“ATFD”), or evaporated by spray drying.

The distillation process can be performed at atmospheric pressure or reduced pressure. Preferably the solvent is removed at a pressure of about 760 mm Hg or less, more preferably at about 400 mm Hg or less, still more preferably at about 80 mm Hg or less, and most preferably from about 30 to about 80 mm Hg.

Spontaneous crystallization refers to crystallization without the help of an external aid such as seeding, cooling etc., and forcible crystallization refers to crystallization with the help of an external aid.

Forcible crystallization may be initiated by a method usually known in the art such as cooling, seeding, partial removal of the solvent from the solution, by adding an anti-solvent to the solution or a combination thereof.

The pure solid form of (±)-O-desmethylvenlafaxine salts obtained in step-(d) may be recovered by conventional techniques known in the art such as filtration, filtration under vacuum, decantation, and centrifugation, or a combination thereof.

In one embodiment, the pure solid form of (±)-O-desmethylvenlafaxine salts can be isolated by filtration employing a filtration media of, for example, a silica gel or celite.

The pure solid form of (±)-O-desmethylvenlafaxine salts obtained by above process may be further dried in, for example, Vacuum Tray Dryer, Rotocon Vacuum Dryer, Vacuum Paddle Dryer or pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In an embodiment, the drying can be carried out at atmospheric pressure or reduced pressures, such as below about 200 mm Hg, or below about 50 mm Hg, at temperatures such as about 35° C. to about 70° C. The drying can be carried out for any desired time period that achieves the desired result, such as times about 1 to 20 hours. Drying may also be carried out for shorter or longer periods of time depending on the product specifications. Temperatures and pressures will be chosen based on the volatility of the solvent being used and the foregoing should be considered as only a general guidance. Drying can be suitably carried out in a tray dryer, vacuum oven, air oven, or using a fluidized bed drier, spin flash dryer, flash dryer and the like. Drying equipment selection is well within the ordinary skill in the art.

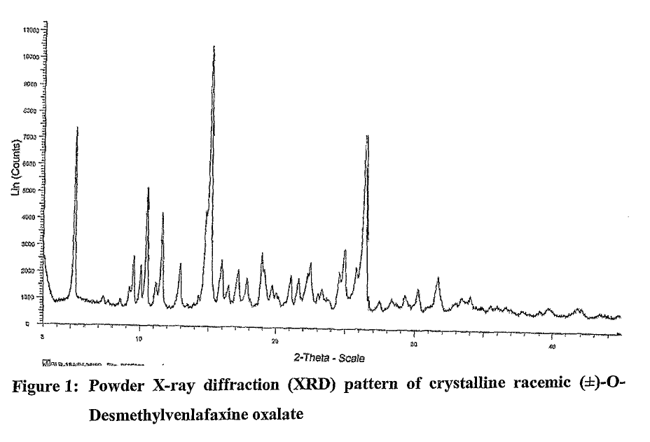

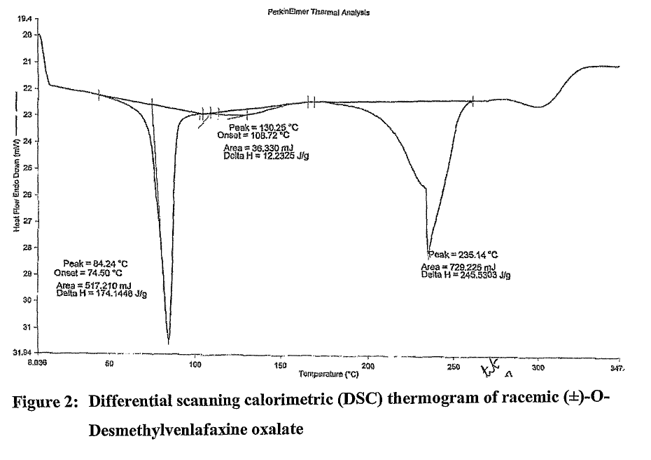

According to another aspect of the present invention, there is provided a solid form of (±)-O-desmethylvenlafaxine oxalate salt characterized by at least one, and preferably all, of the following properties:
i) a powder X-ray diffraction pattern substantially in accordance with FIG. 1;
ii) a powder X-ray diffraction pattern having peaks at about 5.23, 10.49, 11.61, 15.09 and 26.42±0.2 degrees 2-theta substantially as depicted in FIG. 1;
iii) a powder X-ray diffraction pattern having additional peaks at about 9.50, 10.06, 11.16, 12.93, 14.78, 15.94, 17.14, 17.81, 18.95, 21.06, 21.65, 22.49, 24.63, 24.97, 25.83 and 31.68±0.2 degrees 2-theta substantially as depicted in FIG. 1; and
iv) a differential scanning calorimetric (DSC) thermogram substantially in accordance with FIG. 2.

According to another aspect of the present invention, a process for the preparation of solid form of (±)-O-desmethylvenlafaxine oxalate salt is provided, which comprises:
a) providing a solution of (±)-O-desmethylvenlafaxine free base in a suitable solvent or a mixture of suitable solvents;
b) combining the solution obtained in step-(a) with oxalic acid; and
c) optionally, heating the reaction mass obtained in step-(b) to form a clear solution;
d) optionally, substantially removing the solvent from the solution obtained in step-(b) or step-(c) to obtain a residue and dissolving the residue in a suitable solvent with the proviso that the solvent is having less polarity than the solvent used in step-(a);
e) isolating crystalline (±)-O-desmethylvenlafaxine oxalate salt from the solution.

The process can produce crystalline (±)-O-desmethylvenlafaxine oxalate salt in substantially pure form.

The term “substantially pure crystalline (±)-O-desmethylvenlafaxine oxalate salt” refers to the crystalline (±)-O-desmethylvenlafaxine oxalate salt having purity greater than about 98%, specifically greater than about 99%, more specifically greater than about 99.5% and still more specifically greater than about 99.9% (measured by HPLC).

The crystalline (±)-O-desmethylvenlafaxine oxalate salt is stable, consistently reproducible and has good flow properties, and which is particularly suitable for bulk preparation and handling, and so, the novel crystalline (±)-O-desmethylvenlafaxine oxalate salt is suitable for formulating (±)-O-desmethylvenlafaxine. Moreover, the crystalline (±)-O-desmethylvenlafaxine oxalate salt is useful intermediate in the preparation of (±)-O-desmethylvenlafaxine or a pharmaceutically acceptable salt thereof in high purity.

In a preferred embodiment, the crystalline (±)-O-desmethylvenlafaxine oxalate salt obtained according the present invention having water content of about 0.5% to about 15% by weight, specifically about 5% to about 12% by weight, and more specifically about 8.5-10.5% by weight.

The suitable solvent is selected from the group comprising water, alcohols, ketones, chlorinated hydrocarbons, hydrocarbons, nitriles, esters, ethers, polar aprotic solvents, and mixtures thereof. Preferable solvents are alcohols, chlorinated hydrocarbons, hydrocarbons and mixtures thereof, and most preferably methanol, ethanol, isopropanol, tert-butanol, methylene chloride, and mixtures thereof.

Step-(a) of providing a solution of (±)-O-desmethylvenlafaxine free base includes dissolving any form of (±)-O-desmethylvenlafaxine free base (solid or residue) in the suitable solvent, or obtaining an existing solution from a previous processing step.

Preferably the (±)-O-desmethylvenlafaxine free base is dissolved in the solvent at a temperature of about 0° C. to about the reflux temperature of the solvent used, more preferably at about 25° C. to about 100° C., and still more preferably at about 25° C. to about 80° C.

Alternatively, the solution in step-(a) may be prepared by treating an acid addition salt of (±)-O-desmethylvenlafaxine with a base to liberate (±)-O-desmethylvenlafaxine free base and dissolving the (±)-O-desmethylvenlafaxine free base in the suitable solvent.

As acid addition salts, the salts derived from a therapeutically acceptable acid such as hydrochloric acid, hydrobromic acid, sulfuric acid, nitric acid, phosphoric acid, acetic acid, propionic acid, oxalic acid, succinic acid, maleic acid, fumaric acid, benzenesulfonic acid, toluenesulfonic acid, citric acid, glutaric acid, citraconic acid, glutaconic acid, and tartaric acid can be used. More preferable acid addition salt of (±)-O-desmethylvenlafaxine is fumarate salt.

The treatment of an acid addition salt with base is carried out in any solvent and the selection of solvent is not critical. A wide variety of solvents such as chlorinated solvents, hydrocarbon solvents, ether solvents, alcohol solvents, ketone solvents, ester solvents etc., can be used.

The base can be inorganic or organic. Preferable base is an inorganic base selected from ammonia, alkali metal hydroxides, carbonates and bicarbonates. Preferable alkali metal is sodium or potassium.

The oxalic acid in step-(b) may be used directly or in the form of oxalic acid dissolved in a suitable solvent. The suitable solvent used for dissolving oxalic acid is selected from the group comprising water, alcohols, ketones, chlorinated hydrocarbons, hydrocarbons, nitriles, esters, cyclic ethers, aliphatic ethers, polar aprotic solvents, and mixtures thereof.

The combining of the solution with oxalic acid in step-(b) is done in a suitable order, for example, the solution is added to the oxalic acid, or alternatively, the oxalic acid is added to the solution. The addition is carried out drop wise, in one portion, or in more than one portion. In one embodiment, addition is carried out at a temperature of below about 60° C. for at least 15 minutes, and more specifically at a temperature of about 15° C. to about 35° C. from about 20 minutes to about 2 hours. After completion of the addition process, the resulting mass is stirred for at least 20 minutes, more specifically about 30 minutes to about 16 hours, at a temperature of about 20° C. to about 35° C.

The heating in step-(c) is carried out at a temperature of about 40° C. to the reflux temperature of the solvent used for at least 20 minutes, and more preferably at a temperature of about 40° C. to about 80° C. from about 30 minutes to about 4 hours.

The solution obtained in step-(b) or step-(c) can be optionally subjected to carbon treatment or silica gel treatment. The carbon treatment or silica gel treatment can be carried out by methods known in the art, for example by stirring the solution with finely powdered carbon or silica gel at a temperature of below about 70° C. for at least 15 minutes, preferably at a temperature of about 40° C. to about 70° C. for at least 30 minutes; and filtering the resulting mixture through hyflo to obtain a filtrate containing (±)-O-desmethylvenlafaxine oxalate by removing charcoal or silica gel. Preferably, finely powdered carbon is an active carbon. Preferable mesh size of silica gel is 60-120 mesh.

Usually, about 1 to 2 moles, specifically, about 1 to 1.1 moles of oxalic acid is used per 1 mole of (±)-O-desmethylvenlafaxine free base.

Removal of solvent in step-(d) is accomplished by, for example, substantially complete evaporation of the solvent, concentrating the solution and filtering the solid under inert atmosphere. Alternatively, the solvent may also be removed by evaporation. Evaporation can be achieved at sub-zero temperatures by the lyophilisation or freeze-drying technique. The solution may also be completely evaporated in, for example, a pilot plant Rota vapor, a Vacuum Paddle Dryer or in a conventional reactor under vacuum above about 720 mm Hg by flash evaporation techniques by using an agitated thin film dryer (“ATFD”), or evaporated by spray drying.

The distillation process can be performed at atmospheric pressure or reduced pressure at a temperature of about 35° C. to about 70° C. Preferably the solvent is removed at a pressure of about 760 mm Hg or less, more preferably at about 400 mm Hg or less, still more preferably at about 80 mm Hg or less, and most preferably from about 30 to about 80 mm Hg.

The dissolution of the residue in the solvent in step-(d) is carried out at a temperature of above about 20° C., more preferably at about 25° C. to about 100° C., and still more preferably at about 25° C. to about 80° C.

The isolation of pure crystalline (±)-O-desmethylvenlafaxine oxalate salt in step-(e) may be carried out by forcible or spontaneous crystallization.

Spontaneous crystallization refers to crystallization without the help of an external aid such as seeding, cooling etc., and forcible crystallization refers to crystallization with the help of an external aid.

Forcible crystallization may be initiated by a method usually known in the art such as cooling, seeding, partial removal of the solvent from the solution, by adding an anti-solvent to the solution, or a combination thereof.

In an embodiment, the crystallization is carried out by stirring the solution at a temperature of below 30° C. for at least 20 minutes, and more preferably at about 0° C. to about 30° C. from about 1 hour to about 15 hours.

The pure solid form of (±)-O-desmethylvenlafaxine oxalate salt obtained in step-(e) may be recovered by conventional techniques known in the art such as filtration, filtration under vacuum, decantation, and centrifugation, or a combination thereof.

In one embodiment, the pure solid form of (±)-O-desmethylvenlafaxine oxalate salt can be isolated by filtration employing a filtration media of, for example, a silica gel or celite.

The pure solid form of (±)-O-desmethylvenlafaxine oxalate salt obtained by above process may be further dried in, for example, Vacuum Tray Dryer, Rotocon Vacuum Dryer, Vacuum Paddle Dryer or pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In an embodiment, the drying can be carried out at atmospheric pressure or reduced pressures, such as below about 200 mm Hg, or below about 50 mm Hg, at temperatures such as about 35° C. to about 70° C. The drying can be carried out for any desired time period that achieves the desired result, such as times about 1 to 20 hours. Drying may also be carried out for shorter or longer periods of time depending on the product specifications. Temperatures and pressures will be chosen based on the volatility of the solvent being used and the foregoing should be considered as only a general guidance. Drying can be suitably carried out in a tray dryer, vacuum oven, air oven, or using a fluidized bed drier, spin flash dryer, flash dryer and the like. Drying equipment selection is well within the ordinary skill in the art.

The total purity of the solid form of (±)-O-desmethylvenlafaxine oxalate salt obtained by the process disclosed herein is of greater than about 99%, specifically greater than about 99.5%, and more specifically greater than about 99.95% as measured by HPLC.

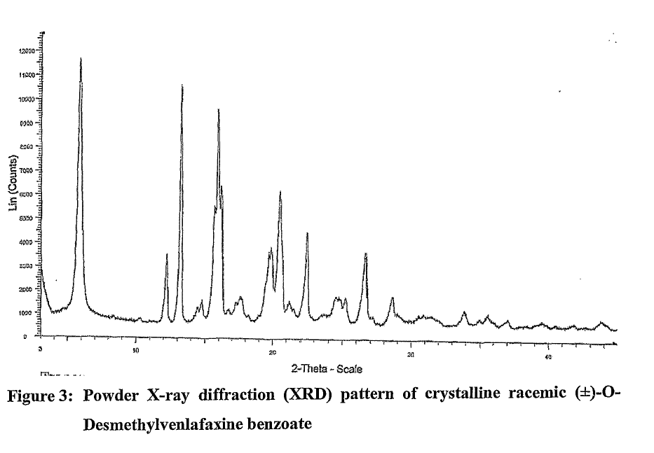

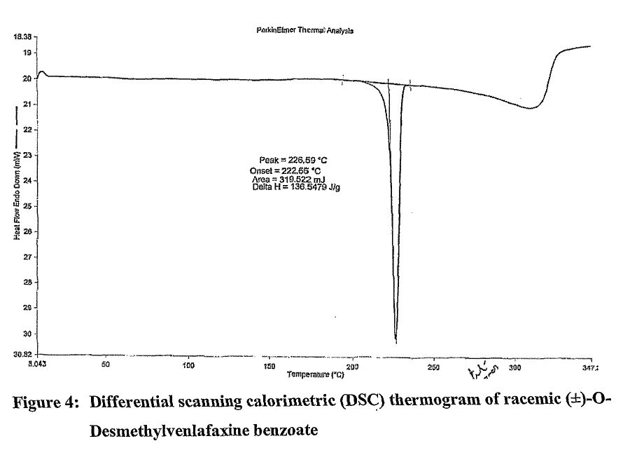

According to another aspect of the present invention, there is provided a solid form of (±)-O-desmethylvenlafaxine benzoate salt characterized by at least one, and preferably all, of the following properties:
i) a powder X-ray diffraction pattern substantially in accordance with FIG. 3;
ii) a powder X-ray diffraction pattern having peaks at about 5.81, 12.16, 13.22, 15.90 and 20.46±0.2 degrees 2-theta substantially as depicted in FIG. 3;
iii) a powder X-ray diffraction pattern having additional peaks at about 14.74, 15.66, 16.14, 17.65, 19.40, 19.73, 19.85, 22.42, 24.53, 24.81, 25.23, 26.65 and 28.62±0.2 degrees 2-theta substantially as depicted in FIG. 3; and
iv) a differential scanning calorimetric (DSC) thermogram in accordance with FIG. 4.

According to another aspect of the present invention, a process for the preparation of solid form of (±)-O-desmethylvenlafaxine benzoate salt is provided, which comprises:
a) providing a solution of (±)-O-desmethylvenlafaxine free base in a suitable solvent or a mixture of suitable solvents;
b) combining the solution obtained in step-(a) with benzoic acid; and
c) optionally, heating the reaction mass obtained in step-(b) to form a clear solution;
d) optionally, substantially removing the solvent from the solution obtained in step-(b) or step-(c) to obtain a residue and dissolving the residue in a suitable solvent with the proviso that the solvent is having less polarity than the solvent used in step-(a);
e) isolating crystalline (±)-O-desmethylvenlafaxine benzoate salt from the solution.

The process can produce crystalline (±)-O-desmethylvenlafaxine benzoate salt in substantially pure form.

The term “substantially pure crystalline (±)-O-desmethylvenlafaxine benzoate salt” refers to the crystalline (±)-O-desmethylvenlafaxine benzoate salt having purity greater than about 98%, specifically greater than about 99%, more specifically greater than about 99.5% and still more specifically greater than about 99.9% (measured by HPLC).

The crystalline (±)-O-desmethylvenlafaxine benzoate salt is stable, consistently reproducible and has good flow properties, and which is particularly suitable for bulk preparation and handling, and so, the novel crystalline (±)-O-desmethylvenlafaxine benzoate salt is suitable for formulating (±)-O-desmethylvenlafaxine. Moreover, the crystalline (±)-O-desmethylvenlafaxine benzoate salt is useful intermediate in the preparation of (±)-O-desmethylvenlafaxine or a pharmaceutically acceptable salt thereof in high purity.

In a preferred embodiment, the crystalline (±)-O-desmethylvenlafaxine benzoate salt obtained according the present invention having water content less than about 5% by weight, specifically less than about 1% by weight, and more specifically less than about 0.3% by weight, and still more specifically is essentially free from water.

The suitable solvent is selected from the group comprising water, alcohols, ketones, chlorinated hydrocarbons, hydrocarbons, nitriles, esters, ethers, polar aprotic solvents, and mixtures thereof. Preferable solvents are alcohol solvents, and most preferably methanol, ethanol, isopropanol, tert-butanol, and mixtures thereof.

Step-(a) of providing a solution of (±)-O-desmethylvenlafaxine free base includes dissolving any form of (±)-O-desmethylvenlafaxine free base (solid or residue) in the suitable solvent, or obtaining an existing solution from a previous processing step.

Preferably the (±)-O-desmethylvenlafaxine free base is dissolved in the solvent at a temperature of about 0° C. to about the reflux temperature of the solvent used, more preferably at about 25° C. to about 100° C., and still more preferably at about 25° C. to about 80° C.

Alternatively, the solution in step-(a) may be prepared by treating an acid addition salt of (±)-O-desmethylvenlafaxine with a base to liberate (±)-O-desmethylvenlafaxine free base and dissolving the (±)-O-desmethylvenlafaxine free base in the suitable solvent.

As acid addition salts, the salts derived from a therapeutically acceptable acid such as hydrochloric acid, hydrobromic acid, sulfuric acid, nitric acid, phosphoric acid, acetic acid, propionic acid, oxalic acid, succinic acid, maleic acid, fumaric acid, benzenesulfonic acid, toluenesulfonic acid, citric acid, glutaric acid, citraconic acid, glutaconic acid, and tartaric acid can be used. More preferable acid addition salt of (±)-O-desmethylvenlafaxine is fumarate salt.

The treatment of an acid addition salt with base is carried out in any solvent and the selection of solvent is not critical. A wide variety of solvents such as chlorinated solvents, hydrocarbon solvents, ether solvents, alcohol solvents, ketone solvents, ester solvents etc., can be used.

The base can be inorganic or organic. Preferable base is an inorganic base selected from ammonia, alkali metal hydroxides, carbonates and bicarbonates. Preferable alkali metal is sodium or potassium.

The benzoic acid in step-(b) may be used directly or in the form of benzoic acid dissolved in a suitable solvent. The suitable solvent used for dissolving benzoic acid is selected from the group comprising water, alcohols, ketones, chlorinated hydrocarbons, hydrocarbons, nitriles, esters, cyclic ethers, aliphatic ethers, polar aprotic solvents, and mixtures thereof.

The combining of the solution with benzoic acid in step-(b) is done in a suitable order, for example, the solution is added to the benzoic acid, or alternatively, the benzoic acid is added to the solution. The addition is carried out drop wise, in one portion, or in more than one portion. In one embodiment, addition is carried out at a temperature of below about 60° C. for at least 15 minutes, and more specifically at a temperature of about 15° C. to about 35° C. from about 20 minutes to about 2 hours. After completion of the addition process, the resulting mass is stirred for at least 20 minutes, more specifically about 30 minutes to about 16 hours, at a temperature of about 20° C. to about 35° C.

The heating in step-(c) is carried out at a temperature of about 40° C. to the reflux temperature of the solvent used for at least 20 minutes, and more preferably at a temperature of about 40° C. to about 80° C. from about 30 minutes to about 4 hours.

The solution obtained in step-(b) or step-(c) can be optionally subjected to carbon treatment or silica gel treatment. The carbon treatment or silica gel treatment can be carried out by methods known in the art, for example by stirring the solution with finely powdered carbon or silica gel at a temperature of below about 70° C. for at least 15 minutes, preferably at a temperature of about 40° C. to about 70° C. for at least 30 minutes; and filtering the resulting mixture through hyflo to obtain a filtrate containing (±)-O-desmethylvenlafaxine benzoate by removing charcoal or silica gel. Preferably, finely powdered carbon is an active carbon. Preferable mesh size of silica gel is 60-120 mesh.

Usually, about 1 to 2 moles, specifically, about 1 to 1.1 moles of benzoic acid is used per 1 mole of (±)-O-desmethylvenlafaxine free base.

Removal of solvent in step-(d) is accomplished by, for example, substantially complete evaporation of the solvent, concentrating the solution and filtering the solid under inert atmosphere. Alternatively, the solvent may also be removed by evaporation. Evaporation can be achieved at sub-zero temperatures by the lyophilisation or freeze-drying technique. The solution may also be completely evaporated in, for example, a pilot plant Rota vapor, a Vacuum Paddle Dryer or in a conventional reactor under vacuum above about 720 mm Hg by flash evaporation techniques by using an agitated thin film dryer (“ATFD”), or evaporated by spray drying.

The distillation process can be performed at atmospheric pressure or reduced pressure at a temperature of about 35° C. to about 70° C. Preferably the solvent is removed at a pressure of about 760 mm Hg or less, more preferably at about 400 mm Hg or less, still more preferably at about 80 mm Hg or less, and most preferably from about 30 to about 80 mm Hg.

The dissolution of the residue in the solvent in step-(d) is carried out at a temperature of above about 20° C., more preferably at about 25° C. to about 100° C., and still more preferably at about 25° C. to about 80° C.

The isolation of pure crystalline (±)-O-desmethylvenlafaxine benzoate salt in step-(e) may be carried out by forcible or spontaneous crystallization.

Spontaneous crystallization refers to crystallization without the help of an external aid such as seeding, cooling etc., and forcible crystallization refers to crystallization with the help of an external aid.

Forcible crystallization may be initiated by a method usually known in the art such as cooling, seeding, partial removal of the solvent from the solution, by adding an anti-solvent to the solution, or a combination thereof.

In an embodiment, the crystallization is carried out by stirring the solution at a temperature of below 30° C. for at least 20 minutes, and more preferably at about 0° C. to about 30° C. from about 1 hour to about 15 hours.

The pure solid form of (±)-O-desmethylvenlafaxine benzoate salt obtained in step-(e) may be recovered by conventional techniques known in the art such as filtration, filtration under vacuum, decantation, and centrifugation, or a combination thereof.

In one embodiment, the pure solid form of (±)-O-desmethylvenlafaxine benzoate salt can be isolated by filtration employing a filtration media of, for example, a silica gel or celite.

The pure solid form of (±)-O-desmethylvenlafaxine benzoate salt obtained by above process may be further dried in, for example, Vacuum Tray Dryer, Rotocon Vacuum Dryer, Vacuum Paddle Dryer or pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In an embodiment, the drying can be carried out at atmospheric pressure or reduced pressures, such as below about 200 mm Hg, or below about 50 mm Hg, at temperatures such as about 35° C. to about 70° C. The drying can be carried out for any desired time period that achieves the desired result, such as times about 1 to 20 hours. Drying may also be carried out for shorter or longer periods of time depending on the product specifications. Temperatures and pressures will be chosen based on the volatility of the solvent being used and the foregoing should be considered as only a general guidance. Drying can be suitably carried out in a tray dryer, vacuum oven, air oven, or using a fluidized bed drier, spin flash dryer, flash dryer and the like. Drying equipment selection is well within the ordinary skill in the art.

The total purity of the solid form of (±)-O-desmethylvenlafaxine benzoate salt obtained by the process disclosed herein is of greater than about 99%, specifically greater than about 99.5%, and more specifically greater than about 99.95% as measured by HPLC.

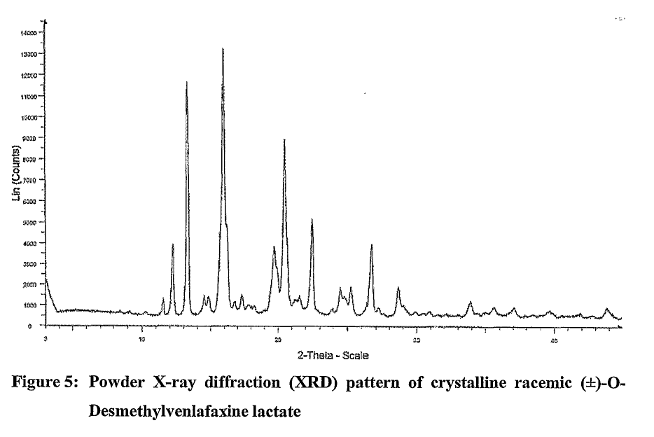

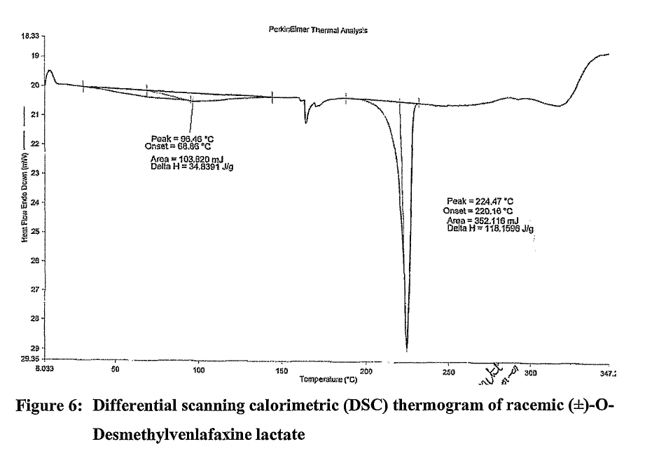

According to another aspect of the present invention, there is provided a solid form of (±)-O-desmethylvenlafaxine lactate salt characterized by at least one, and preferably all, of the following properties:
i) a powder X-ray diffraction pattern substantially in accordance with FIG. 5;
ii) a powder X-ray diffraction pattern having peaks at about 12.20, 13.28, 15.95, 16.20 and 19.72±0.2 degrees 2-theta substantially as depicted in FIG. 5;
iii) a powder X-ray diffraction pattern having additional peaks at about 11.51, 14.50, 17.31, 19.94, 20.49, 22.48, 24.51, 24.80, 25.27, 26.72 and 28.68±0.2 degrees 2-theta substantially as depicted in FIG. 5; and
iv) a differential scanning calorimetric (DSC) thermogram substantially in accordance with FIG. 6.

According to another aspect of the present invention, a process for the preparation of solid form of (±)-O-desmethylvenlafaxine lactate salt is provided, which comprises:
a) providing a solution of (±)-O-desmethylvenlafaxine free base in a suitable solvent or a mixture of suitable solvents;
b) combining the solution obtained in step-(a) with lactic acid; and
c) optionally, heating the reaction mass obtained in step-(b) to form a clear solution;
d) optionally, substantially removing the solvent from the solution obtained in step-(b) or step-(c) to obtain a residue and dissolving the residue in a suitable solvent with the proviso that the solvent is having less polarity than the solvent used in step-(a);
e) isolating crystalline (±)-O-desmethylvenlafaxine lactate salt from the solution.

The process can produce crystalline (±)-O-desmethylvenlafaxine lactate salt in substantially pure form.

The term “substantially pure crystalline (±)-O-desmethylvenlafaxine lactate salt” refers to the crystalline (±)-O-desmethylvenlafaxine lactate salt having purity greater than about 98%, specifically greater than about 99%, more specifically greater than about 99.5% and still more specifically greater than about 99.9% (measured by HPLC).

The crystalline (±)-O-desmethylvenlafaxine lactate salt is stable, consistently reproducible and has good flow properties, and which is particularly suitable for bulk preparation and handling, and so, the novel crystalline (±)-O-desmethylvenlafaxine lactate salt is suitable for formulating (±)-O-desmethylvenlafaxine. Moreover, the crystalline (±)-O-desmethylvenlafaxine lactate salt is useful intermediate in the preparation of (±)-O-desmethylvenlafaxine or a pharmaceutically acceptable salt thereof in high purity.

In a preferred embodiment, the crystalline (±)-O-desmethylvenlafaxine lactate salt obtained according the present invention having water content of about 0.5% to about 15% by weight, specifically about 5% to about 12% by weight, and more specifically about 7.5-9.5% by weight.

The suitable solvent is selected from the group comprising water, alcohols, ketones, chlorinated hydrocarbons, hydrocarbons, nitriles, esters, ethers, polar aprotic solvents, and mixtures thereof. Preferable solvents are alcohols, ethers, chlorinated hydrocarbons, nitriles and mixtures thereof, and most preferably methanol, ethanol, isopropanol, tert-butanol, methylene chloride, diisopropyl ether, acetonitrile and mixtures thereof.

Step-(a) of providing a solution of (±)-O-desmethylvenlafaxine free base includes dissolving any form of (±)-O-desmethylvenlafaxine free base (solid or residue) in the suitable solvent, or obtaining an existing solution from a previous processing step.

Preferably the (±)-O-desmethylvenlafaxine free base is dissolved in the solvent at a temperature of about 0° C. to about the reflux temperature of the solvent used, more preferably at about 25° C. to about 100° C., and still more preferably at about 25° C. to about 80° C.

Alternatively, the solution in step-(a) may be prepared by treating an acid addition salt of (±)-O-desmethylvenlafaxine with a base to liberate (±)-O-desmethylvenlafaxine free base and dissolving the (±)-O-desmethylvenlafaxine free base in the suitable solvent.

As acid addition salts, the salts derived from a therapeutically acceptable acid such as hydrochloric acid, hydrobromic acid, sulfuric acid, nitric acid, phosphoric acid, acetic acid, propionic acid, oxalic acid, succinic acid, maleic acid, fumaric acid, benzenesulfonic acid, toluenesulfonic acid, citric acid, glutaric acid, citraconic acid, glutaconic acid, and tartaric acid can be used. More preferable acid addition salt of (±)-O-desmethylvenlafaxine is fumarate salt.

The treatment of an acid addition salt with base is carried out in any solvent and the selection of solvent is not critical. A wide variety of solvents such as chlorinated solvents, hydrocarbon solvents, ether solvents, alcohol solvents, ketone solvents, ester solvents etc., can be used.

The base can be inorganic or organic. Preferable base is an inorganic base selected from ammonia, alkali metal hydroxides, carbonates and bicarbonates. Preferable alkali metal is sodium or potassium.

The lactic acid in step-(b) may be used directly or in the form of lactic acid dissolved in a suitable solvent. The suitable solvent used for dissolving lactic acid is selected from the group comprising water, alcohols, ketones, chlorinated hydrocarbons, hydrocarbons, nitriles, esters, cyclic ethers, aliphatic ethers, polar aprotic solvents, and mixtures thereof.

The combining of the solution with lactic acid in step-(b) is done in a suitable order, for example, the solution is added to the lactic acid, or alternatively, the lactic acid is added to the solution. The addition is carried out drop wise, in one portion, or in more than one portion. In one embodiment, addition is carried out at a temperature of below about 60° C. for at least 15 minutes, and more specifically at a temperature of about 15° C. to about 35° C. from about 20 minutes to about 2 hours. After completion of the addition process, the resulting mass is stirred for at least 20 minutes, more specifically about 30 minutes to about 16 hours, at a temperature of about 20° C. to about 35° C.

The heating in step-(c) is carried out at a temperature of about 40° C. to the reflux temperature of the solvent used for at least 20 minutes, and more preferably at a temperature of about 40° C. to about 80° C. from about 30 minutes to about 4 hours.

The solution obtained in step-(b) or step-(c) can be optionally subjected to carbon treatment or silica gel treatment. The carbon treatment or silica gel treatment can be carried out by methods known in the art, for example by stirring the solution with finely powdered carbon or silica gel at a temperature of below about 70° C. for at least 15 minutes, preferably at a temperature of about 40° C. to about 70° C. for at least 30 minutes; and filtering the resulting mixture through hyflo to obtain a filtrate containing (±)-O-desmethylvenlafaxine lactate by removing charcoal or silica gel. Preferably, finely powdered carbon is an active carbon. Preferable mesh size of silica gel is 60-120 mesh.

Usually, about 1 to 2 moles, specifically, about 1 to 1.1 moles of lactic acid is used per 1 mole of (±)-O-desmethylvenlafaxine free base.

Removal of solvent in step-(d) is accomplished by, for example, substantially complete evaporation of the solvent, concentrating the solution and filtering the solid under inert atmosphere. Alternatively, the solvent may also be removed by evaporation. The solution may also be completely evaporated in, for example, a pilot plant Rota vapor, a Vacuum Paddle Dryer or in a conventional reactor under vacuum above about 720 mm Hg by flash evaporation techniques by using an agitated thin film dryer (“ATFD”), or evaporated by spray drying.

The distillation process can be performed at atmospheric pressure or reduced pressure at a temperature of about 35° C. to about 70° C. Preferably the solvent is removed at a pressure of about 760 mm Hg or less, more preferably at about 400 mm Hg or less, still more preferably at about 80 mm Hg or less, and most preferably from about 30 to about 80 mm Hg.

The dissolution of the residue in the solvent in step-(d) is carried out at a temperature of above about 20° C., more preferably at about 25° C. to about 100° C., and still more preferably at about 25° C. to about 80° C.

The isolation of pure crystalline (±)-O-desmethylvenlafaxine lactate salt in step-(e) may be carried out by forcible or spontaneous crystallization.

Spontaneous crystallization refers to crystallization without the help of an external aid such as seeding, cooling etc., and forcible crystallization refers to crystallization with the help of an external aid.

Forcible crystallization may be initiated by a method usually known in the art such as cooling, seeding, partial removal of the solvent from the solution, by adding an anti-solvent to the solution, or a combination thereof.

In an embodiment, the crystallization is carried out by stirring the solution at a temperature of below 30° C. for at least 20 minutes, and more preferably at about 0° C. to about 30° C. from about 1 hour to about 15 hours.

The pure solid form of (±)-O-desmethylvenlafaxine lactate salt obtained in step-(e) may be recovered by conventional techniques known in the art such as filtration, filtration under vacuum, decantation, and centrifugation, or a combination thereof.

In one embodiment, the pure solid form of (±)-O-desmethylvenlafaxine lactate salt can be isolated by filtration employing a filtration media of, for example, a silica gel or celite.

The pure solid form of (±)-O-desmethylvenlafaxine lactate salt obtained by above process may be further dried in, for example, Vacuum Tray Dryer, Rotocon Vacuum Dryer, Vacuum Paddle Dryer or pilot plant Rota vapor, to further lower residual solvents. Drying can be carried out under reduced pressure until the residual solvent content reduces to the desired amount such as an amount that is within the limits given by the International Conference on Harmonization of Technical Requirements for Registration of Pharmaceuticals for Human Use (“ICH”) guidelines.

In an embodiment, the drying can be carried out at atmospheric pressure or reduced pressures, such as below about 200 mm Hg, or below about 50 mm Hg, at temperatures such as about 35° C. to about 70° C. The drying can be carried out for any desired time period that achieves the desired result, such as times about 1 to 20 hours. Drying may also be carried out for shorter or longer periods of time depending on the product specifications. Temperatures and pressures will be chosen based on the volatility of the solvent being used and the foregoing should be considered as only a general guidance. Drying can be suitably carried out in a tray dryer, vacuum oven, air oven, or using a fluidized bed drier, spin flash dryer, flash dryer and the like. Drying equipment selection is well within the ordinary skill in the art.

The total purity of the solid form of (±)-O-desmethylvenlafaxine lactate salt obtained by the process disclosed herein is of greater than about 99%, specifically greater than about 99.5%, and more specifically greater than about 99.95% as measured by HPLC.

(±)-O-Desmethylvenlafaxine free base or an acid addition salt thereof used as starting material can be obtained by processes described in the prior art, for example by the process described in the U.S. Pat. No. 4,535,186.

Karl Fisher analysis, which is well known in the art, is also used to determine the quantity of water in a sample.

In one embodiment, the substantially pure solid state forms of (±)-O-desmethylvenlafaxine salts disclosed herein for use in the pharmaceutical compositions of the present invention, wherein 90 volume-percent of the particles (D₉₀) have a size of less than or equal to about 500 microns, specifically less than or equal to about 300 microns, more specifically less than or equal to about 200 microns, still more specifically less than or equal to about 100 microns, and most specifically less than or equal to about 15 microns.

In another embodiment, the particle sizes of substantially pure solid state forms of (±)-O-desmethylvenlafaxine salts can be achieved via comminution, or a mechanical process of reducing the size of particles which includes any one or more of cutting, chipping, crushing, milling, grinding, micronizing, trituration or other particle size reduction methods known in the art, to bring the solid state forms the desired particle size range.

According to another aspect of the present invention, there is provided a method for the use of novel solid forms of (±)-O-desmethylvenlafaxine salts for the treatment of patients suffering from depression (e.g., major depressive disorder, bipolar disorder, and dysthymia), anxiety, panic disorder, generalized anxiety disorder, post traumatic stress disorder, attention deficit disorder, obsessive compulsive disorder, schizophrenia, obesity, anorexia nervosa, cognitive enhancement, cognitive impairment, and cessation of smoking; comprising administering the novel solid forms of (±)-O-desmethylvenlafaxine salts, or a pharmaceutical composition that comprises novel solid forms of (±)-O-desmethylvenlafaxine salts, along with pharmaceutically acceptable excipients.

According to another aspect of the present invention, there is provided pharmaceutical compositions comprising the solid state forms of (±)-O-desmethylvenlafaxine salts and one or more pharmaceutically acceptable excipients.

According to another aspect of the present invention, there is provided pharmaceutical compositions comprising the solid state forms of (±)-O-desmethylvenlafaxine salts prepared according to processes of the present invention in any of its embodiments and one or more pharmaceutically acceptable excipients.

According to another aspect of the present invention, there is provided a process for preparing a pharmaceutical formulation comprising combining any one of the solid state forms of (±)-O-desmethylvenlafaxine salts prepared according to processes of the present invention in any of its embodiments, with one or more pharmaceutically acceptable excipients.

Yet another embodiment of the present invention is directed to pharmaceutical compositions comprising at least a therapeutically effective amount of any one of the substantially pure solid state forms of (±)-O-desmethylvenlafaxine salts of the present invention. Such pharmaceutical compositions may be administered to a mammalian patient in any dosage form, e.g., liquid, powder, elixir, injectable solution, etc. Dosage forms may be adapted for administration to the patient by oral, buccal, parenteral, ophthalmic, rectal and transdermal routes or any other acceptable route of administration. Oral dosage forms include, but are not limited to, tablets, pills, capsules, troches, sachets, suspensions, powders, lozenges, elixirs and the like. The solid state forms of (±)-O-desmethylvenlafaxine salts of the present invention may also be administered as suppositories, ophthalmic ointments and suspensions, and parenteral suspensions, which are administered by other routes. The dosage forms may contain any one of the solid state forms of (±)-O-desmethylvenlafaxine salts of the present invention as is or, alternatively, may contain any one of the solid state forms of (±)-O-desmethylvenlafaxine salts of the present invention as part of a composition. The pharmaceutical compositions may further contain one or more pharmaceutically acceptable excipients. Suitable excipients and the amounts to use may be readily determined by the formulation scientist based upon experience and consideration of standard procedures and reference works in the field, e.g., the buffering agents, sweetening agents, binders, diluents, fillers, lubricants, wetting agents and disintegrants described hereinabove.

In another embodiment of the present invention, there is provided pharmaceutical compositions comprising crystalline (±)-O-desmethylvenlafaxine oxalate salt and one or more pharmaceutically acceptable excipients.

In another embodiment of the present invention, there is provided pharmaceutical compositions comprising crystalline (±)-O-desmethylvenlafaxine benzoate salt and one or more pharmaceutically acceptable excipients.

In another embodiment of the present invention, there is provided pharmaceutical compositions comprising crystalline (±)-O-desmethylvenlafaxine lactate salt and one or more pharmaceutically acceptable excipients.

Capsule dosages will contain the solid state forms of (±)-O-desmethylvenlafaxine salts of the present invention within a capsule which may be coated with gelatin. Tablets and powders may also be coated with an enteric coating. The enteric-coated powder forms may have coatings containing at least phthalic acid cellulose acetate, hydroxypropylmethyl cellulose phthalate, polyvinyl alcohol phthalate, carboxy methyl ethyl cellulose, a copolymer of styrene and maleic acid, a copolymer of methacrylic acid and methyl methacrylate, and like materials, and if desired, they may be employed with suitable plasticizers and/or extending agents. A coated capsule or tablet may have a coating on the surface thereof or may be a capsule or tablet comprising a powder or granules with an enteric-coating.

Tableting compositions may have few or many components depending upon the tableting method used, the release rate desired and other factors. For example, the compositions of the present invention may contain diluents such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols like mannitol and sorbitol, acrylate polymers and copolymers, as well as pectin, dextrin and gelatin.

Other excipients contemplated by the present invention include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

The following examples are provided to enable one skilled in the art to practice the invention and are merely illustrate the process of this invention. However, it is not intended in any way to limit the scope of the present invention.

### EXAMPLES

### Example 1

### Preparation of Solid Form of (±)-O-Desmethylvenlafaxine Oxalate

(±)-O-Desmethylvenlafaxine free base (5.0 g) was dissolved in isopropanol (50 ml) at 25-30° C. The solution was followed by the addition of oxalic acid (2.39 g) at 25-30° C. and the solution was stirred at 25-30° C. for 12 hours. Solvent was evaporated under vacuum at 40-45° C. and the residue was stirred with methylene chloride (50 ml) for 5-6 hours at 25-30° C. The precipitated product was filtered and washed with dichloromethane (5 ml). The product was dried under vacuum at 55-60° C. to yield 6.3 g of (±)-O-desmethylvenlafaxine oxalate salt as a crystalline solid [HPLC Purity: 99.76%, Water content (by KF): 9.7% w/w].

### Example 2

### Preparation of Solid Form of (±)-O-Desmethylvenlafaxine Benzoate

(±)-O-Desmethylvenlafaxine free base (5.0 g) was dissolved in isopropanol (50 ml) at 25-30° C. The solution was followed by the addition of benzoic acid (2.32 g) at 25-30° C. and the solution was stirred for 12 hours at 25-30° C. The precipitated product was filtered and washed with isopropanol (5 ml). The product was dried under vacuum at 55-60° C. to yield 1.92 g of (±)-O-desmethylvenlafaxine benzoate salt as a crystalline solid [HPLC Purity: 99.9%, Water content (by KF): 0.88% w/w].

### Example 3

### Preparation of Solid Form of (±)-O-Desmethylvenlafaxine Lactate

(±)-O-Desmethylvenlafaxine free base (5.0 g) was dissolved in isopropanol (50 ml) at 25-30° C. The solution was followed by the addition of lactic acid (1.86 g) at 25-30° C. and the solution was stirred at 25-30° C. for 15 hours. The precipitated product was filtered and washed with isopropanol (5 ml). The product was dried under vacuum at 55-60° C. to yield 1.88 g of (±)-O-desmethylvenlafaxine lactate salt as a crystalline solid [HPLC Purity: 99.2%, Water content (by KF): 8.08% w/w].

1. Solid state form of a salt of (±)-1-[2-(dimethylamino)-1-(4-hydroxyphenyl)ethyl]cyclohexanol [(±)-O-desmethylvenlafaxine salt], wherein the salt of (±)-O-desmethylvenlafaxine is an oxalate salt, a benzoate salt or a lactate salt.

2. The solid state (±)-O-desmethylvenlafaxine salt of claim 1, which is in a crystalline form or in an amorphous form; and wherein the solid state form is anhydrous and/or solvent-free form or a hydrate and/or a solvate form.

3. The solid state (±)-O-desmethylvenlafaxine salt of claim 1, having the following characteristics, wherein:
a) the solid state form of (±)-O-desmethylvenlafaxine oxalate salt is characterized by one or more of the following properties:
i) a powder X-ray diffraction pattern substantially in accordance with FIG. 1;
ii) a powder X-ray diffraction pattern having peaks at about 5.23, 10.49, 11.61, 15.09 and 26.42±0.2 degrees 2-theta;
iii) a powder X-ray diffraction pattern having additional peaks at about 9.50, 10.06, 11.16, 12.93, 14.78, 15.94, 17.14, 17.81, 18.95, 21.06, 21.65, 22.49, 24.63, 24.97, 25.83 and 31.68±0.2 degrees 2-theta; and
iv) a differential scanning calorimetric (DSC) thermogram substantially in accordance with FIG. 2;
b) the solid state form of (±)-O-desmethylvenlafaxine benzoate salt is characterized by one or more of the following properties:
i) a powder X-ray diffraction pattern substantially in accordance with FIG. 3;
ii) a powder X-ray diffraction pattern having peaks at about 5.81, 12.16, 13.22, 15.90 and 20.46±0.2 degrees 2-theta;
iii) a powder X-ray diffraction pattern having additional peaks at about 14.74, 15.66, 16.14, 17.65, 19.40, 19.73, 19.85, 22.42, 24.53, 24.81, 25.23, 26.65 and 28.62±0.2 degrees 2-theta; and
iv) a differential scanning calorimetric (DSC) thermogram substantially in accordance with FIG. 4; and
c) the solid state form of (±)-O-desmethylvenlafaxine lactate salt is characterized by one or more of the following properties:
i) a powder X-ray diffraction pattern substantially in accordance with FIG. 5;
ii) a powder X-ray diffraction pattern having peaks at about 12.20, 13.28, 15.95, 16.20 and 19.72±0.2 degrees 2-theta;
iii) a powder X-ray diffraction pattern having additional peaks at about 11.51, 14.50, 17.31, 19.94, 20.49, 22.48, 24.51, 24.80, 25.27, 26.72 and 28.68±0.2 degrees 2-theta; and
iv) a differential scanning calorimetric (DSC) thermogram substantially in accordance with FIG. 6.

4. A process for the preparation of solid (±)-O-desmethylvenlafaxine salt of any one of claims 1 to 3, comprising:
a) providing a solution of (±)-O-desmethylvenlafaxine free base in a solvent selected from the group consisting of water, an alcohol, a ketone, a chlorinated hydrocarbon, a hydrocarbon, a nitrile, an ester, an ether, a polar aprotic solvent, and mixtures thereof;
b) combining the solution obtained in step-(a) with an acid selected from the group consisting of oxalic acid, benzoic acid and lactic acid; and
c) optionally, heating the reaction mass obtained in step-(b) to form a clear solution;
d) optionally, substantially removing the solvent from the solution obtained in step-(b) or step-(c) to obtain a residue followed by dissolving the residue in a solvent with the proviso that the solvent is having less polarity than the solvent used in step-(a);
e) isolating solid state form of (±)-O-desmethylvenlafaxine salt from the solution, obtained in step-(b), step-(c) or step-(d), by forcible or spontaneous crystallization.

5.-6. (canceled)

7. The process of claim 4, wherein the solvent is selected from the group consisting of water, methanol, ethanol, isopropanol, tert-butanol, methylene chloride, diisopropyl ether, acetonitrile, and mixtures thereof.

8. The process of claim 4, wherein the acid in step-(b) is used directly or in the form of the acid dissolved in a suitable solvent; wherein the acid in step-(b) is used in a molar ratio of about 1 to 2 moles per 1 mole of (±)-O-desmethylvenlafaxine free base; wherein the combining of the solution with the acid in step-(b) is carried out by adding the acid to the solution of (±)-O-desmethylvenlafaxine free base or by adding the (±)-O-desmethylvenlafaxine free base solution to the acid; wherein the reaction mass in step-(c) is heated at a temperature of about 40° C. to the reflux temperature of the solvent used for at least 20 minutes; and wherein the solution obtained in step-(b) or step-(c) is optionally subjected to carbon treatment or silica gel treatment.

9. The process of claim 8, wherein the acid is used in a molar ratio of about 1 to 1.1 moles per 1 mole of (±)-O-desmethylvenlafaxine free base; wherein the addition in step-(b) is carried out at a temperature of below about 60° C. for at least 15 minutes; and wherein the reaction mass obtained after addition of the acid in step-(b) is further stirred for at least 20 minutes at a temperature of about 20° C. to about 35° C.

10.-11. (canceled)

12. The process of claim 4, wherein the removal of solvent in step-(d) is accomplished by evaporation, vacuum drying, spray drying, freeze drying or a combination thereof; wherein the crystallization in step-(e) is initiated by cooling, seeding, partial removal of the solvent from the solution, by adding an anti-solvent to the solution or a combination thereof.

13. (canceled)

14. The process of claim 4, wherein the crystallization in step-(e) is carried out by stirring the solution at a temperature of below 30° C. for about 30 minutes to about 15 hours; wherein the solid form of (±)-O-desmethylvenlafaxine salt obtained in step-(e) is recovered by filtration, filtration under vacuum, decantation, and centrifugation, or a combination thereof; wherein the pure solid form of (±)-O-desmethylvenlafaxine salt obtained in step-(e) is further dried under atmospheric pressure or reduced pressures at a temperature of about 35° C. to about 70° C.; and wherein the solid form of (±)-O-desmethylvenlafaxine salt obtained has a total purity of about 99% to about 99.9%.

15. (canceled)

16. The solid state (±)-O-desmethylvenlafaxine salt of claim 1, wherein the solid form of (±)-O-desmethylvenlafaxine oxalate salt has a water content of about 0.5% to about 15% by weight; and wherein the solid form of (±)-O-desmethylvenlafaxine lactate salt has a water content of about 0.5% to about 15% by weight.

17. The solid state (±)-O-desmethylvenlafaxine salt of claim 16, wherein the (±)-O-desmethylvenlafaxine oxalate salt has a water content of about 8.5% to about 10.5% by weight; and wherein the solid form of (±)-O-desmethylvenlafaxine lactate salt has a water content of about 7.5% to about 9.5% by weight.

18.-86. (canceled)

87. A pharmaceutical composition comprising a solid state form of (±)-O-desmethylvenlafaxine salt and one or more pharmaceutically acceptable excipients, wherein the salt of (±)-O-desmethylvenlafaxine is an oxalate salt, a benzoate salt or a lactate salt; and wherein the pharmaceutical composition is prepared by a process comprising combining the solid state form of (±)-O-desmethylvenlafaxine salt with one or more pharmaceutically acceptable excipients.

88.-92. (canceled)

93. The pharmaceutical composition of claim 87, wherein the solid state form of (±)-O-desmethylvenlafaxine salt has a D₉₀ particle size of less than or equal to about 500 microns.

94. The pharmaceutical composition of claim 93, wherein the D₉₀ particle size is less than or equal to about 400 microns; less than or equal to about 200 microns; less than or equal to about 100 microns; less than or equal to about 60 microns; or less than or equal to about 15 microns.

95.-98. (canceled)
